(19) United States
(12) Patent Application Publication
(10) Pub. No.: US 2015/0157655 A1
(43) Pub. Date: Jun. 11, 2015

(54) SELENIUM CONTAINING NUCLEOSIDES AS NUTRITIONAL SUPPLEMENTS

(71) Applicant: SeNA Research, Inc., Marietta, GA (US)

(72) Inventor: Zhen Huang, Marietta, GA (US)

(73) Assignee: SeNA Research, Inc., Marietta, GA (US)

(21) Appl. No.: 14/411,444

(22) PCT Filed: May 29, 2013
(86) PCT No.: PCT/US13/43114

§ 371 Date: Dec. 26, 2014

Related U.S. Application Data
(60) Provisional application No. 61/666,381, filed on Jun. 29, 2012.

(51) Int. Cl. A61K 31/7072

(52) U.S. Cl. CPC A61K 31/7072

(57) ABSTRACT

Nutritional supplements are described comprising selenium containing nucleosides. Methods of making the nutritional supplements and using the nutritional supplements are also described.

### GOVERNMENT RIGHTS

Development of the inventions described herein was at least partially funded with government support through a National Science Foundation grant (MCB-0517092) and the U.S. government has certain rights in the inventions.

### BACKGROUND

Selenium as a trace mineral is essential to good health but required only in small amounts. Selenium is incorporated into proteins to make selenoproteins, which are important antioxidant enzymes. Though selenium is most noticeable for its antioxidant properties, the biological activities of selenium are not just limited to its antioxidant abilities. For example some selenoproteins help regulate thyroid function and play a role in the immune system.

Studies have found that although human selenium deficiency is rare in the U.S, other countries, most notably China, where soil concentration of selenium is low, is known to have the highest rate of selenium deficiency. Selenium deficiency is attributed to the development of a form of heart disease, hypothyroidism, a weakened immune system, making the body more susceptible to illnesses caused by other nutritional, biochemical or infectious stresses, and more specifically to three diseases, i.e. Keshan Disease, Kashin-Beck Disease, as well as Myxedematous Endemic Cretinism.

Selenium deficiency generally is seen in people who rely on total parenteral nutrition (TPN) as their sole source of nutrition, people with severe gastrointestinal problems, such as Crohn's disease, or with surgical removal of part of the stomach, or people with iodine deficiency.

National Institute of Health (NIH) provides a dietary supplement fact sheet for Selenium, which is incorporated herein by reference. According to the NIH Selenium fact sheet, Recommended Dietary Allowances (RDA) for selenium, in micrograms (mcg) per day, for children and adults is between 20 mcg to 70 mcg/day as listed in Table 1 below.

TABLE 1

| Age | Males and Females | Pregnancy | Lactation |
|---|---|---|---|
| (years) | (mcg/day) | (mcg/day) | (mcg/day) |
| 1-3 | 20 | N/A | N/A |
| 4-8 | 30 | N/A | N/A |
| 9-13 | 40 | N/A | N/A |
| 14-18 | 55 | 60 | 70 |
| 19+ | 55 | 60 | 70 |

High blood levels of selenium (greater than 100 mcg/dL) can result in a condition called selenosis with symptoms of gastrointestinal upsets, hair loss, white blotchy nails, garlic breath odor, fatigue, irritability, and mild nerve damage. Tolerable upper intake level (UL) for selenium as listed in Table 2 below was set to prevent the risk of toxicity associated high blood level selenium.

TABLE 2

| Age | | Males and Females (mcg/day) |
|---|---|---|
| 0-6 | months | 45 |
| 7-12 | months | 60 |
| 1-3 | years | 90 |
| 4-8 | years | 150 |
| 9-13 | years | 280 |
| 14-18 | years | 400 |
| 19+ | years | 400 |

Selenium supplement intake has been associated with the reduced incidence of prostate cancer, colorectal cancer, and lung cancer, may help to prevent coronary artery disease, to relieve symptoms of arthritis by controlling levels of free radicals or to prolong the lives of HIV-positive men and women.

### DETAILED DESCRIPTION OF PREFERRED EMBODIMENTS

The selenium containing nucleosides (SeNA) described herein provide additional or alternative selenium source as nutritional supplements compared to conventional selenium supplements such as selenides, selenomethionine, or high selenium yeasts. The SeNA in discussion is generally represented by formula I below, where R₂, R₃, and R₄ are each independently selected from the group consisting of H, OH, and SeCH₃ and R_{Nu}, is selected from the group consisting of adenine, 8-selenoadenine, thymine, 2-selenothymine, 4-selenothymine, 5-alkylselenothymidine, uracil, 2-selenouracil, 4-selenouracil, 5-alkylselenouracil, guanine, 6-selenoguanine, 8-selenoguanine, cytosine, 2-selenocytosine, and 5-alkylselenocytosine.

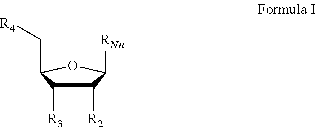

The synthesis of these SeNA has been reported in Nucleosides, Nucleotides and Nucleic Acids, 28:1, 56-66 by Lin et al. entitled “Facile Synthesis and Anti-Tumor Cell Actively of Se-Containing Nucleosides” (hereinafter the Lin reference) and in U.S. provisional patent application serial number 61/083,815 to Huang et al., entitled “Se-Containing Nucleosides, Methods of Synthesis, and Anti-cancer Activity of Such,” both incorporated herein by reference. Examplatory SeNA 1, 2, and 3 are shown in formula II below, where for SeNA1: R₁═H, R₂═MeSe—, R₃═OH, R₄═OH; for SeNA2, R₁═CH₃, R₂═H, R₃═MeSe—, R₄═OH, and for SeNA3, R₁═CH₃, R₂═H, R₃═OH, R₄═MeSe—.

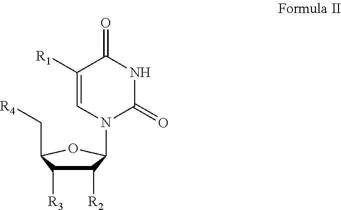

These SeNA compounds are able to cross cell walls to provide inhibitory action against at least prostate tumor cells as disclosed in the Lin reference. SeNAs have also been reported to provide interesting properties to DNA and RNA when incorporated into nucleotide chains, for example to improve translation accuracy and/or efficiencies of tRNA as disclosed in Chemistry and Biodiversity Vol. 5 (2008) pg 396-407 to Caton-Williams et al. entitled “Biochemistry of Selenium-Dereivatized Naturally Occurring and Unnatural Nucleic Acids”, in Chemistry and Biodiversity, 2010, 7, 753-785 to Sheng et al. entitled “Selenium Derivatization of Nucleic Acids for X-ray Crystal Structure and Function Studies”, and in Chem. Soc. Rev., 2011, 40, 4591-4602 to Lin et al. entitled “Nucleic Acid X-ray Crystallograph via Direct Selenium Derivatization”, all three incorporated herein by reference.

Nutritional selenium supplements is known to contain selenium in difference chemical forms, including selenide, selenomethionine, and high selenium yeast preparations etc. The selenium supplement product types, quality, and safety for example have been discussed in Journal of the American College of Nutrition, Vol. 20, No. 1, 1-4 (2001) to Schrauzer entitled “Nutritional Selenium Supplements: Product Types, Quality, and Safety”, incorporated herein by reference. Schrauzer favors selenomethionine over selenide and has recommended 200 mcg/day selenium intake.

Among the currently commercial available selenium supplements, high selenium yeast such as SelenoExcell® has been reported to provide a variety of organo selenium nutrients that were integrated into Baker's yeast cells. During the discussion entitled “Selenium and Cancer: what the research shows” by Passwater et al, incorporated herein by reference, it has been disclosed that the variety of organo selenium nutrients provided in the high selenium yeast is believed to be the source of its alleged anti-cancer activities. Passwater et al. however mainly discussed selenium integrated into proteins or as amino acids. Passwater et al. failed to recognize the potential impacts of SeNA or selenium's impact on nucleosides.

Selenium containing nucleosides, especially SeNA discussed herein therefore is recognized here as a previously untapped source as an additional or alternative selenium source for nutritional supplement. The SeNA compounds are believed to provide nutritional and health benefit that could compliment or extends beyond current commercial selenium supplements. These SeNA compounds are believed to be bioavailable to provide unique nutritional values. For example, to provide interesting and useful properties to DNA and RNA when incorporated into nucleotide chains, to improve DNA polymerization (or replication) and RNA transcription fidelity, to improve translation accuracy and/or efficiencies of tRNA, or to simply providing an additional or alternative source of selenium.

The SeNA compound discussed herein can be formulated alone or together with other nutritional supplement compounds including other commercially available selenium compounds. Because the SeNA compounds are produced by synthetic methods, relatively accurate amount of selenium in the SeNA compounds can be estimated or measured. A nutritional supplement containing the SeNA compounds can be formulated in many forms, e.g., liquid, solid, gel, emulsion, powder, tablet, capsule, or gel cap (e.g., soft or hard gel cap). The recommended dietary allowances and tolerable upper intake level outlined in tables 1 and 2 above provides guidance with the amount of SeNA to be included in the supplements. Any of the nutritional supplement formulations can be prepared using well known methods by those having ordinary skill in the art, e.g., by mixing the recited ingredients in the proper amounts. Ingredients for inclusion in a nutritional supplement formulation are generally commercially available, e.g., from Douglas Labs, Inc., B&D Nutritional Ingredients, Stella Labs, Nutricap Labs, Sedona Labs, Pharmline Inc., and Garden State Nutritionals.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein.

What is claimed is:

1. A nutritional supplement comprising selenium containing nucleosides in Formula I 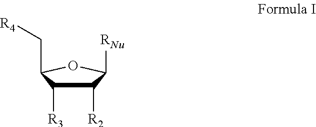
where R9, R₃, and R₄ are each independently selected from the group consisting of H, OH, and SeCH₃ and RNu is selected from the group consisting of adenine, 8-selenoadenine, thymine, 2-selenothymine, 4-selenothymine, 5-alkylselenothymidine, uracil, 2-selenouracil, 4-selenouracil, 5 -alkylselenouracil, guanine, 6-selenoguanine, 8-selenoguanine, cytosine, 2-selenocytosine, and 5-alkylselenocytosine.

2. A nutritional supplement comprising selenium containing nucleic acid in Formula II 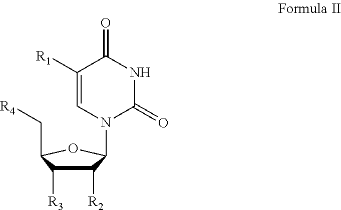
where for R₁═H, R₂═MeSe—, R₃═OH, R₄═OH; R₁═CH₃, R₂═H, R₃═MeSe—, R₄═OH, or R₁═CH₃, R₂═H, R₃═OH, R₄═MeSe—.

3. A method of making nutritional supplement that comprises selenium containing nucleosides in Formula I 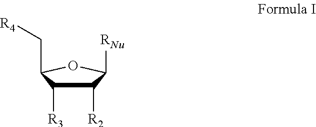
where R₂, R₃, and R₄ are each independently selected from the group consisting of OH, and SeCH₃ and RNu is selected from the group consisting of adenine, 8-selenoadenine, thymine, 2-selenothymine, 4-selenothymine, 5-alkylselenothymidine, uracil, 2-selenouracil, 4-selenouracil, 5-alkylselenouracil, guanine, 6-selenoguanine, 8-selenoguanine, cytosine, 2-selenocytosine, and 5-alkylselenocytosine.

4. A method of making nutritional supplement that comprises selenium containing nucleic acid in Formula H 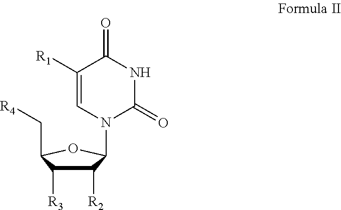
where for R₁═H, R₂═MeSe—, R₃═OH, R₄═OH; R₁═CH₃, R₂═H, R₃═MeSe—, R₄═OH, or R₁═CH₃, R₂═H, R₃═OH, R₄═MeSe—.

5. A method of using nutritional supplement that comprises selenium containing nucleosides in Formula I to treat or prevent cancer, to prolong lives of HIV positive patients, to improve cardiovascular health, to prevent or treat arthritis 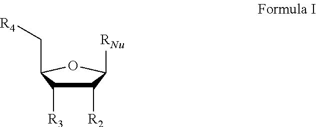
where R₂, R₃, and R₄ are each independently selected from the group consisting of H, OH, and SeCH₃ and RNu is selected from the group consisting of adenine, 8-selenoadenine, thymine, 2-selenothymine, 4-selenothymine, 5-alkylselenothymidine, uracil, 2-selenouracil, selenouracil, 5-alkylselenouracil, guanine, 6-selenoguanine, 8-selenoguanine, cytosine, 2-selenocytosine, and 5-alkylselenocytosine.

6. A method of using nutritional supplement that comprises selenium containing nucleic acid in Formula II to treat or prevent cancer, to prolong lives of HIV positive patients, to improve cardiovascular health, to prevent or treat arthritis 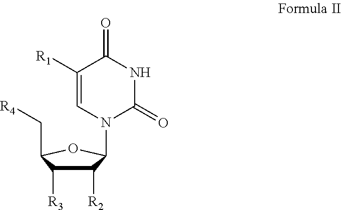
where for R₁═H, R₂═MeSe—, R₃═OH, R₄═OH; R₁═CH₃, R₂═H, R₃═MeSe—, R₄═OH, or R₁═CH₃, R₂═OH, R₃═OH, R₄═MeSe—.
